(19) United States
(12) Patent Application Publication
(10) Pub. No.: US 2016/0106950 A1
(43) Pub. Date: Apr. 21, 2016

(54) FOREHEAD-WEARABLE LIGHT STIMULATOR HAVING ONE OR MORE LIGHT PIPES

(71) Applicant: Curzio Vasapollo, Tokyo (JP)

(72) Inventor: Curzio Vasapollo, Tokyo (JP)

(21) Appl. No.: 14/517,918

(22) Filed: Oct. 19, 2014

(51) Int. Cl. A61M 21/02; A61B 5/00; A61B 5/04; A61B 5/0482; A61B 5/0205

(52) U.S. Cl. CPC A61M 21/02; A61B 5/0482; A61B 5/02055; A61B 5/04012; A61B 5/4812; A61M 2021/0044

(57) ABSTRACT

A forehead-wearable light stimulator having one or more light pipes provides reliable light stimulation to a user. The stimulation can have high intensity and multiple colors. The intensity of the stimulation is less sensitive to device placement than in known light stimulators having no light pipes. The intensity is sufficient to deliver light through the eyelids, and is sufficient for the light's color to be perceived through the eyelids. The light stimulator improves on the state of the art, thereby enabling multiple new applications in the fields of biofeedback, lucid dreaming, and light-based alarms.

### FIELD OF THE INVENTION

The present invention relates to light stimulation devices, and more particularly to wearable light stimulation devices.

### BACKGROUND OF THE INVENTION

Light stimulation is used in biofeedback, lucid dream induction, light therapy, and light-based alarms.

Biofeedback is the process of gaining greater awareness of physiological functions, with the goal of being able to consciously control them. Some of the processes that can be controlled include brainwaves, muscle tone, skin conductance, heart rate, and pain perception. Biofeedback has been shown to provide a viable alternative to pharmaceutical intervention, sometimes with equivalent results. In particular, biofeedback is generally considered effective in the treatment of insomnia and anxiety.

Biofeedback often involves visual feedback. During a typical biofeedback therapy in a specialized clinic, patients observe a visual representation of one or more chosen physiological parameters on a computer monitor. For instance, an animated lotus flower may bloom when the patient relaxes, and close up when the patient becomes less relaxed. Learning to relax one's muscles, slow down one's heart rate, and achieve deep, regular breathing are goals of biofeedback applied to insomnia and anxiety.

A drawback of using a computer image for visual feedback is that the eyes must remain open throughout the biofeedback procedure, impeding relaxation. In the case of insomnia therapy, the requirement for the patient to keep his/her eyes open is even more problematic. It is possible for a patient to learn how to relax and become sleepy while his/her eyes remain open, but transitioning directly into sleep is difficult.

A lucid dream is any dream in which one is aware that one is dreaming. Lucid dreaming can be used for recreation (as it provides a completely convincing virtual reality), therapy, self healing and self discovery (as it allows one to probe and interact with his/her own subconscious through dream characters and dream events), and for problem solving (as lucid dreams constitute a separate state of consciousness, potentially allowing one to process problems in a way different from waking awareness, sometimes discovering novel and creative solutions).

The “light cue” technique for achieving lucidity within a dream is well known. An electronic device delivers a light cue in the form of a blinking, bright light to a dreaming user. This light stimulus is often incorporated within the dream. The dreaming person may see a light house, or car lights, as the brain tries to merge the light stimulus with the ongoing dream scene. By striving to become aware of intermittent or bright lights in waking life, the user can also become aware of such lights inside a dream. The user eventually learns to become lucid within a dream when the cue is delivered.

The Novadreamer™ is a lucid dream induction device that first appeared in the 1980s, and used red LEDs positioned over the eyes of the wearer to deliver the light stimulus. The necessity to place LEDs above the wearer's eyes dictates that this type of device must be a sleep mask. However, sleep masks have drawbacks.

First, they can be uncomfortable if a person is not used to sleeping with a sleep mask. Second, a sleep mask may disturb sleep, because facial movements and twitches move the mask slightly, producing a tactile sensation which can tickle and easily awaken the wearer. Third, when the device circuitry is positioned on the wearer's eyes and nose as in a sleep mask, the only way to detect REM sleep is by analyzing the reflection of infrared light by the eyelid—and this method is prone to error, because the sensor cannot differentiate between subtle mask movements (caused by body movements and facial muscle activity) and actual eye movements.

The Remee™ is a more recently developed lucid dreaming aid, which does away with any type of REM monitoring, and simply delivers the light cue at random or timed intervals throughout the night. Due to its inability to detect REM it yields false negatives and false positives, sometimes not delivering the light cue during a dream, and sometimes awakening the wearer from deep sleep by unnecessarily stimulating the wearer.

The Aurora™ is an EEG-based lucid dreaming aid which promises better REM detection accuracy than infrared based methods. It is a sort of headband worn over the forehead. Due to its placement, it cannot have light emitting components positioned in front of the eyes. Instead, its LEDs are positioned on the device, which is fastened to the forehead, and the LEDs are oriented towards the wearer's eyes. Due to the distance and the uncertainty in the angle between the LEDs and the eyes, the intensity of the perceived light is often greatly reduced. Furthermore, to position the light source at an angle, so that it may be oriented towards the eyes, “through hole” light emitting components are used, their supporting leads bent slightly at manufacturing time. This restricts the light sources to through hole LEDs (whereas most high intensity LEDs used in illumination are solder mount and have no leads). Through hole LEDs also require manual assembly, and lead to higher manufacturing costs. The headband must be precisely positioned on the forehead for the weak light to reach the eyes. If the headband shifts during sleep, light may no longer reach the eyes properly, and the light cue may be missed by the wearer. For these reasons, simply orienting the light source towards the eyes does not produce strong, reliable light stimulation.

Seasonal Affective Disorder (SAD) is a mood disorder experienced in concurrence with periods of low exposure to light, low vitamin D synthesis, and lack of physical activity, conditions which often accompany the winter months. Mayo Clinic states that light therapy is of proven effectiveness for treating seasonal affective disorder and light therapy is seen as its main form of treatment. The effectiveness of light therapy for treating SAD may be linked to the fact that light therapy (consisting of simply exposing the subject to bright light) makes up for lost sunlight exposure and resets the body's internal clock.

Currently, light therapy requires sitting in front of a light box, and is more efficient if the subject is looking straight into the light box (with or without closing his/her eyes) because this increases the intensity of the light. As a consequence, during light therapy, a user is not able to freely engage in most other activities.

It is a well-known fact that light is used by the body to regulate circadian rhythms. In particular, blue light is effective at suppressing melatonin, a sleep hormone. For this reason, light based alarms (sometimes utilizing blue light) have appeared, such as the Lumie Bodyclock™ or the Iwaku™ or the Philips Wake Up Light™, that deliver light of increasing strength prior to a user's wake-up time to facilitate awakening. The main drawbacks of such products are that the light's perceived intensity is limited by their distance from the user, and that they are not aware of the sleeping user's conditions (such as the sleep stage), information that could be used to pick a favorable time to begin awakening the user, thus improving the well-being of the user upon his/her wake.

The Lumi™ mask is a now defunct sleep mask which could deliver light directly into the wearer's eyes. Although superior to the Lumie™ for light intensity, it had the same drawbacks as mask-type lucid dreaming devices, namely comfort and unwanted tactile sensations during sleep. It also did not exploit the fact that it was head-worn to capture physiological signs which would have increased its usefulness by allowing it to pick the best moment to awaken the user.

Blue light is particularly effective at suppressing melatonin, and seems to also be particularly effective at treating depression even when this depression is not related to the season. However, the human eyelid blocks blue light; so that even when using blue light alarm clocks such as the Lumie, the color of the perceived light is not blue.

Several software applications for the Android™ operating system, and the Basis™ wristwatch in the hardware domain use actigraphy to determine a user's sleep stage, and based on this determination pick a favorable time to vibrate or sound an alarm. Unfortunately these products cannot administer a light stimulus.

Patent Appl. No. US20140009282 discloses a personal alarm system which includes an appliance worn near the eye. In the embodiments of FIGS. 6A, 6B, and 6C, light is projected towards the user's eyes in a way similar to the Aurora™ These embodiments, as drawn in the figures, can potentially host high brightness, solder mount LEDs which are not susceptible to the drawbacks of through hole LEDs used in the Aurora™. However, they present the following problems. The first problem is comfort and user-friendliness. The embodiments of FIGS. 6A and 6B require an adhesive surface to removably adhere to the face of the user. Due to sweat, however, the embodiments of FIGS. 6A and 6B would have a high likelihood of becoming displaced throughout the night. The adhesive would have to be replaced. What type of adhesive could be used is not specified. There is generally a trade-off between adhesiveness and safety; the adhesive component (such as acrylic acid) can be increased to provide robust adhesion, but has adverse health effects (from skin sensitivity to respiratory problems) when concentration is increased, particularly when the adhesive is worn for long periods of time, such as throughout the night on a regular basis. The adhesion area available in the embodiments of FIGS. 6A and 6B is not sufficient to achieve safe, reliable adhesion.

The embodiment of FIG. 6C wraps around the ear of the user (6C) like sunglasses, but this presents an even greater obstacle for comfort; the plastic behind the ear will produce pain when the user is sleeping on his/her side.

The second problem in the embodiments of FIGS. 6A, 6B, and 6C is that there is no allowance for EEG, EMG, respiration, and other measurements cited in the disclosure as parameters on which the “wake-up moment” is to be detected. The only physiological signs that could be detected by the head-worn embodiments presented are the heart rate (from reflectance oximetry) and actigraphy, but these are poor metrics for determining the “wake-up moment”.

### SUMMARY

The light stimulator of the invention utilizes one or more light pipes to guide light stimulus to a wearer's eyes. This allows more freedom in the structure and placement of the stimulator on the wearer's head, reduces the need for precise positioning of the stimulator, and removes the need for the light source to be precisely positioned and oriented. The freedom in structure and placement of the stimulator so obtained alleviates problems in the prior art related to adhesion and comfort, thereby enabling the stimulator to be worn comfortably and reliably during sleep.

The light stimulator of the invention improves on the state of the art in several relevant fields.

In the field of biofeedback, the light stimulator of the invention allows light based visual feedback to be delivered without the need for the user to keep his/her eyes open, or wear a mask, or be connected by wires to bulky equipment. This provides insomnia sufferers a comfortable way to practice biofeedback and transition smoothly into sleep.

In the field of lucid dreaming the light stimulator of the invention allows a strong light cue to be delivered reliably without the need for the user to wear a mask over his/her eyes. The stimulator can easily include means for acquiring an EEG signal, because the light-pipe based light delivery system allows the stimulator to be positioned on the forehead. By analyzing an EEG signal, reliable detection of REM sleep is possible according to known methods (for instance by measuring the amplitude of brain waves in the beta region and disappearance of sleep spindles). Accordingly, light stimulation can be initiated at a favorable time, i.e. immediately after entering REM sleep.

In the field of light therapy the light stimulator of the invention provides a way to deliver light stimulation without occluding the user's field of vision entirely, thereby enabling light therapy to be carried out while the user is engaging in other activities, thereby reclaiming productive time. Further, it also provides a way to deliver light therapy while the user's eyes are closed and the user is lying comfortably in bed.

The light stimulator of the invention also improves on light-based wake-up systems, because it can provide light from high brightness light emitting components of the solder mount type, used in illumination, to be directed toward the eye of the user, resulting in perceived luminous intensity that is comparable to that of a sunny sky. Further, the combination of physiological monitoring and a light-based wake-up system can provide the best type of awakening: a light stimulator can choose the best time to awaken the user based on his/her physiological signs, and awaken the user gradually and naturally with simulated sunlight.

One general aspect of the invention is a forehead-wearable light stimulator including: one or more physiological sensors capable of acquiring physiological parameters of a wearer of the light stimulator; a central processing unit capable of receiving the physiological parameters from the one or more physiological sensors, and accordingly providing a light stimulation signal at times and intensities responsive to the physiological parameters; at least one light source capable of receiving the light stimulation signal, and accordingly emitting light; and one or more light pipes capable of conducting the light from the at least one light source to the eyes of the wearer of the light stimulation apparatus.

In some embodiments, the one or more physiological sensors include at least one of: an EEG sensor; a pulse oximetry sensor; a heart rate sensor; a breathing sensor; and a temperature sensor.

In some embodiments, the light stimulation signal is obtained by transforming one or more physiological parameters.

In some embodiments, the light stimulator also includes: a dream onset detector, capable of analyzing the physiological parameters so as to provide a dream onset signal, and wherein the central processing unit provides the light stimulation signal in response to the dream onset signal, so as to induce lucid dreaming.

In some embodiments, the light stimulator also includes: a waking time determiner, capable of determining an ideal waking time, and wherein the luminous stimulation signal is provided at the ideal waking time so as to awaken the wearer.

In some embodiments, the at least one light source is a multi-color light source.

In some embodiments, the at least one light source is substantially of a single color.

Another general aspect of the invention is a forehead-wearable light stimulator including: an EEG sensor capable of acquiring EEG parameters of a wearer of the light stimulator; a central processing unit capable of receiving the EEG parameters from the EEG sensors, and accordingly providing a light stimulation signal at times and intensities responsive to the EEG parameters; at least one light source capable of receiving the light stimulation signal, and accordingly emitting light; and one or more light pipes capable of conducting the light from the at least one light source to the eyes of the wearer of the light stimulation apparatus.

In some embodiments, the EEG sensor is cooperative with at least one of: a pulse oximetry sensor; a heart rate sensor; a breathing sensor; and a temperature sensor.

In some embodiments, the light stimulation signal is obtained by analyzing an EEG signal.

In some embodiments, the light stimulator also includes: a dream onset detector, capable of analyzing the EEG signal so as to provide a dream onset signal, and wherein the central processing unit provides the light stimulation signal in response to the dream onset signal, so as to induce lucid dreaming.

In some embodiments, the light stimulator also includes: a waking time determiner, capable of determining an ideal waking time, and wherein the luminous stimulation signal is provided at the ideal waking time so as to awaken the wearer.

In some embodiments, the at least one light source is a multi-color light source.

In some embodiments, the at least one light source is substantially of a single color.

### BRIEF DESCRIPTION OF THE DRAWINGS

The invention will be more fully understood by reference to the detailed description, in conjunction with the following figures, wherein:

FIG. 1 is a front oblique front view of a light stimulator having two light pipes, the light stimulator being affixed to a user's forehead by means of an electrode patch, the two light pipes carrying light directed towards the user's eye lids.

FIG. 2 is a rear view of the light stimulator of FIG. 1, showing the side that contacts the user, with the electrode patch removed to expose four female snap button connectors, also showing the pulse oximeter window.

FIG. 3 is an oblique rear view of the light stimulator of FIG. 2.

FIG. 4 is a rear oblique view of the light pipes of FIGS. 1, 2, and 3.

FIG. 5 is a bottom oblique view of the light stimulator of FIG. 3, showing the holes into which the light pipes are inserted.

FIG. 6 is a front oblique view of the electrode patch, showing four male snap button connectors, and an opening shaped so as to accommodate the pulse oximeter window.

FIG. 7 is a rear view of the electrode patch, showing four adhesive contact surfaces and the opening shaped so as to accommodate the pulse oximeter window.

FIG. 8 is a rear oblique view of the light stimulator of FIG. 1 also having a supporting headband, wherein the headband is placed on the back of the light stimulator prior to attaching an electrode patch to the light stimulator.

FIG. 9 is a front oblique view of the light stimulator of FIG. 8 affixed to a user's forehead by means of the adhesive electrode patch, and by the supporting headband.

FIG. 10 is a front oblique view of the light stimulator of FIG. 1, showing the light pipes and a respiration sensor.

### DETAILED DESCRIPTION OF THE INVENTION

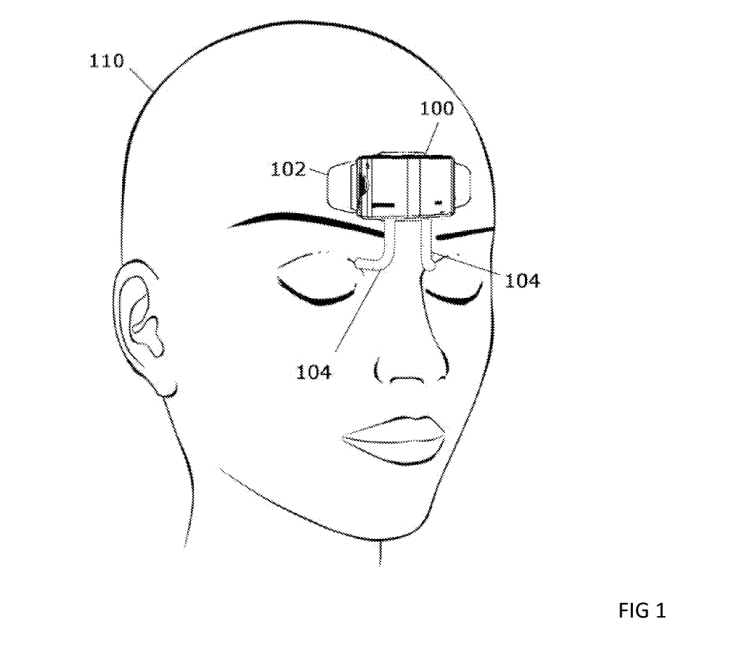

In FIG. 1, a user 110 is shown wearing a light stimulator 100, the stimulator being affixed to the forehead of the user 110 by means of an adhesive electrode patch 102. The light stimulator 100 includes two light pipes 104, which transport light emitted within the apparatus 100 to the user's 110 eye lids.

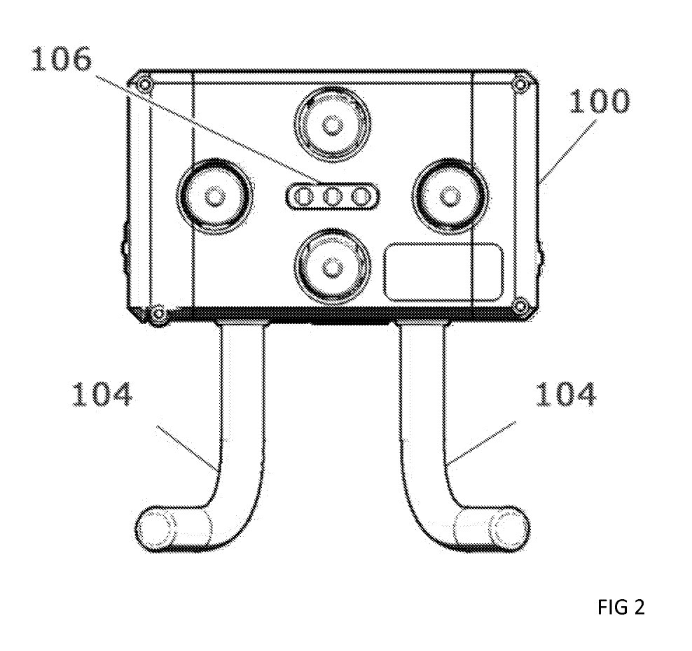

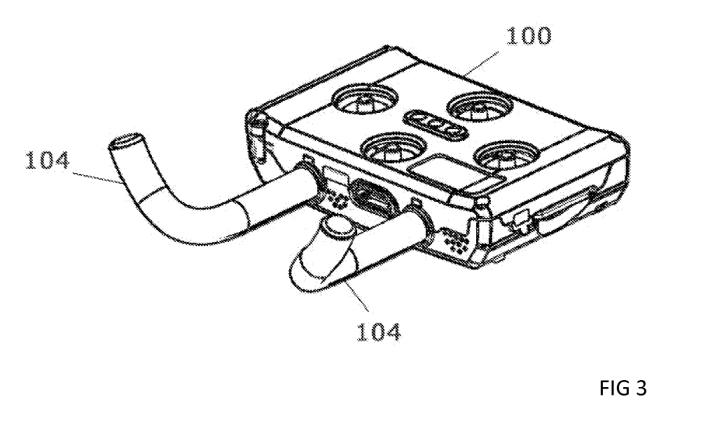

In FIG. 2 and FIG. 3, the light stimulator 100 and light pipes 104 of FIG. 1 are shown in detail from two different angles, with the supporting electrode patch 102 removed for clarity. A pulse oximeter 106 allows monitoring of the heart rate of the user 110.

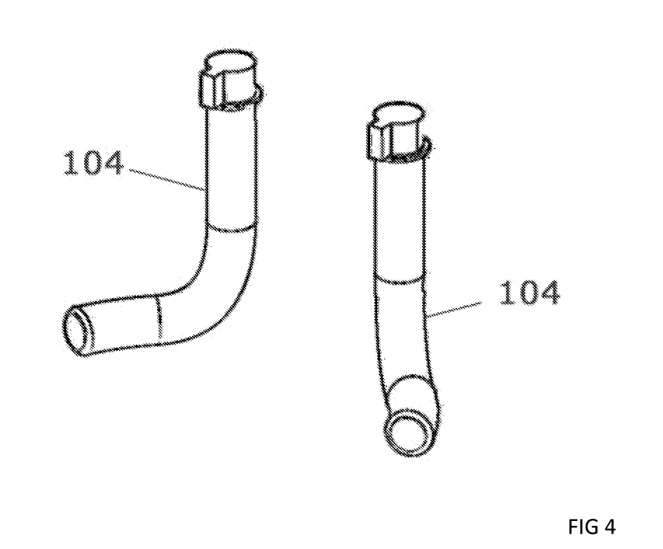

Referring to FIG. 4, the light pipes 104 are removable, and are shown without the main body 100 of the light stimulator.

With reference to FIG. 5, two high-brightness full-color LEDs located inside the stimulator 100 emit light from two holes 500 located at the bottom of the stimulator's 100 enclosure. The transparent light pipes 104 are inserted into these holes. When the stimulator is affixed to the forehead of the user 110, the light pipes 104 direct light emitted by the LEDs towards the eyes. If the light intensity and light pipe transparency are sufficient, light can be strong enough to be perceived through the eyelids. This is a novel way of delivering visual biofeedback to a subject while his/her eyes are closed. For comfort, and since the light pipes are positioned and remain near the wearer's eyes, these light pipes can be made of soft material such as food grade TPE, a low-cost material which can achieve a very high degree of transparency. There are also types of silicon rubber that are used to create soft LED lenses in illumination applications, and these silicon rubbers also have extremely high transparency, and exhibit good total internal reflection. Employing a soft material also allows the light pipes 104 to be plugged into the main body of the stimulator 100, and removed easily for convenience.

To create sufficient luminosity for the light to penetrate the eyelid and be perceived as having a definite color, with the possibility of simulating a sunrise or a bright sunny day, or for blue light therapy, special 5050 size RGB LEDs are used; these LEDs have a forward current of up to 200 mA, and a power dissipation of up to 1.8 W. Due to the high brightness, light intensity always starts low and increases gradually as the stimulation progresses, giving the user an opportunity to close his/her eyelids if the intensity becomes uncomfortable.

Due to the directionality of the light (reaching the eye perpendicularly) and the transparency of the eye, shifts in the position of the stimulator 100 do not translate to significant differences in perceived light intensity. A mask does not need to be used to deliver light, thus eliminating problems inherent in sleep masks such as lack of comfort, tickling sensations, and awakenings.

For biofeedback purposes, the intensity of light which can be delivered by this method is sufficient to create strong color perception even through the eyelids. Therefore, even when the eyes of the subject are closed, a stimulus of varying intensity, color and frequency can be delivered as visual feedback of the physiological sign being monitored. This capability allows a user to deeply relax while undergoing biofeedback therapy, and to practice biofeedback just prior to sleeping, possibly transitioning smoothly into sleep during the procedure. When the user begins to sleep, the stimulator 100 may detect this event by analyzing one or more physiological signs, such as the EEG, and discontinue the stimulus.

In the treatment of Seasonal Affective Disorder, the present invention allows a mask-less, comfortable miniaturized stimulator to be worn throughout sleep, and to begin administering the therapy in the morning, just prior to awakening, thereby accomplishing multiple goals at the same time: slowly awakening the subject with a simulated sunrise, delivering sufficient light to suppress melatonin levels leading to a refreshing awakening, and then utilizing this unproductive time prior to wake-up to carry out light therapy, freeing up the time normally spent in front of a light box by light therapy subjects for other pursuits.

By using high brightness LEDs and very high transparency light pipes, it is possible to deliver blue light of sufficient intensity to be perceived as blue even through the eyelids. This creates a novel and interesting sensation (as blue color is never under natural circumstances perceived when the eyelids are closed), and is also useful from a biofeedback perspective.

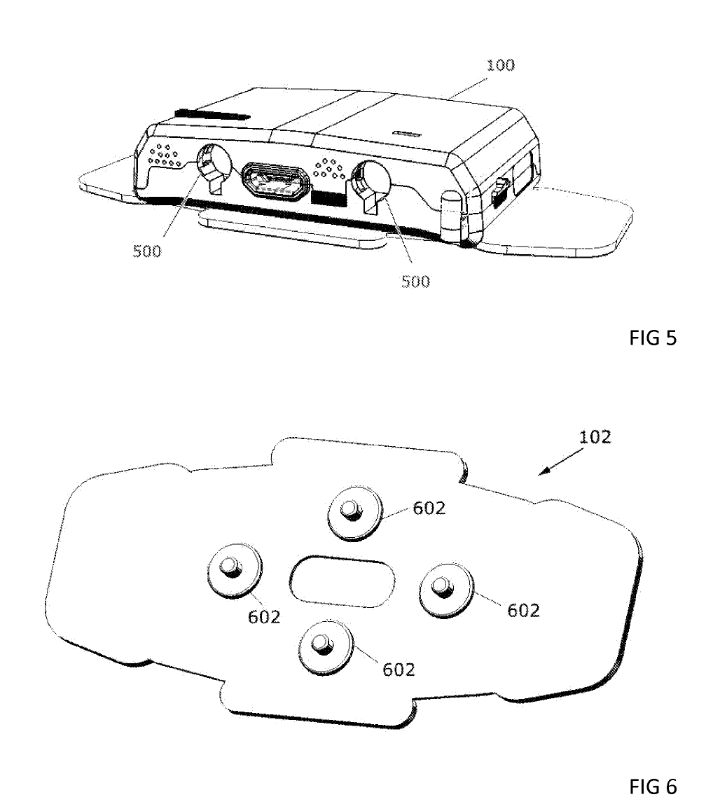

FIG. 6 is a front view of the electrode patch 102 showing the four male snap button connectors 602 by which the electrode patch is electrically and mechanically connected to the light stimulator 100.

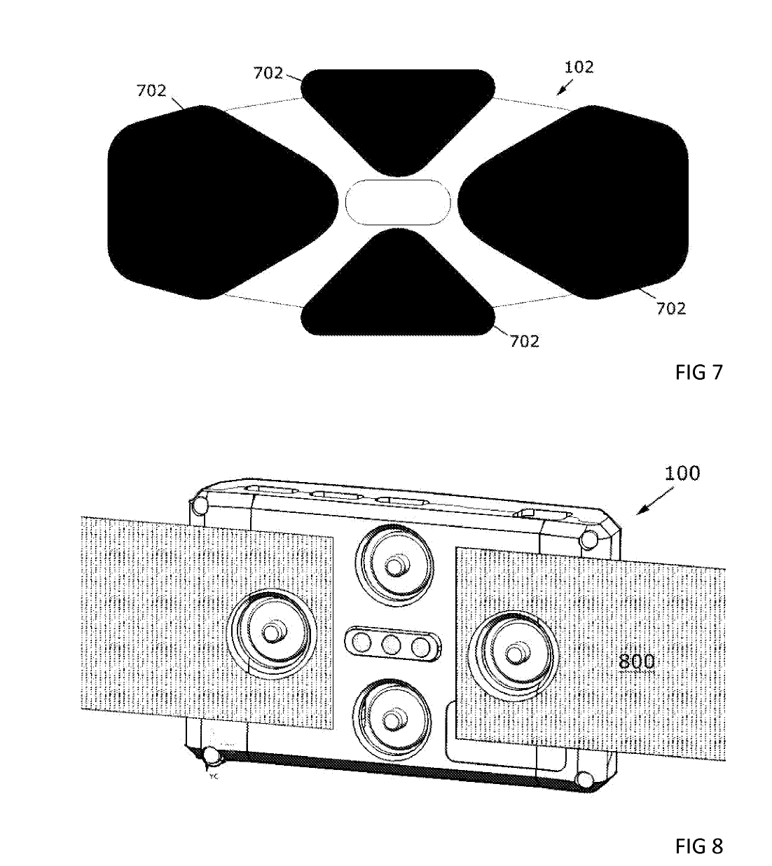

FIG. 7 is a rear view of the electrode patch 102 showing the gel electrodes 702 which allow the light stimulator 100 to both acquire an EEG signal and adhere to the forehead of the user 110.

FIG. 8 is a rear view of the light stimulator 100 and a headband 800 positioned against the light stimulator 100 prior to attaching the electrode patch 102.

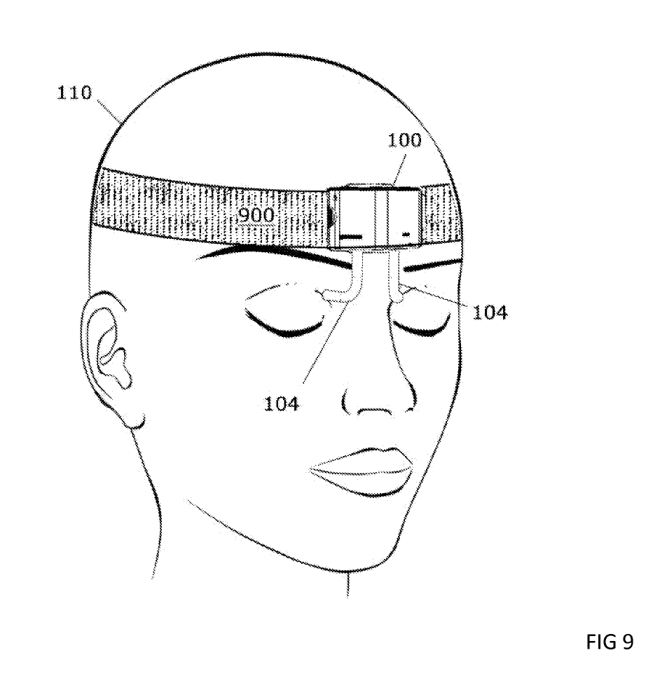

FIG. 9 shows the user 110 wearing the light stimulator 100 with the supporting headband 800.

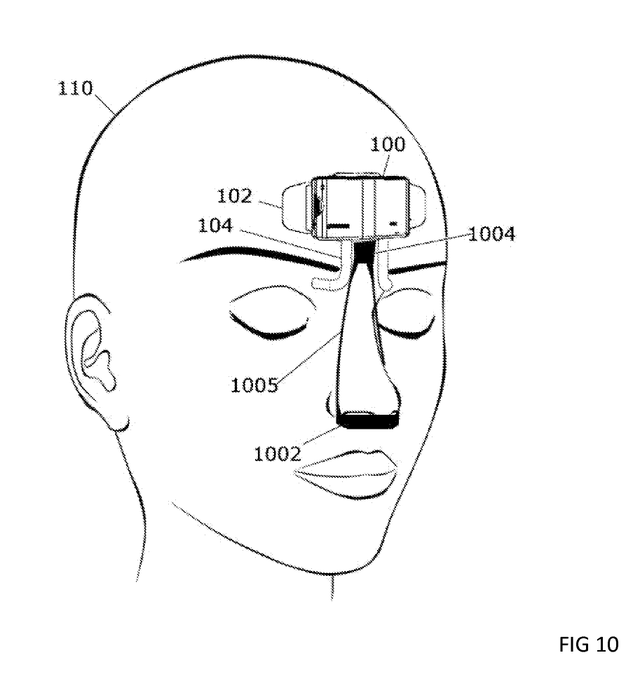

FIG. 10 shows the user 110 wearing an alternate embodiment of the light stimulator 100, this embodiment having a breathing sensor 1002 connected to the light stimulator 100 by a nose harness 1005 and a removable electrical connector 1004.

The transformation of physiological parameters into a visual stimulus for biofeedback is a trivial task for those skilled in the art. Nevertheless a possible method is disclosed here for completeness. The EEG signal acquired by the stimulator 100 via the electrode patch 102 is filtered to obtain the magnitude of the high frequency component above 40 Hz, possibly applying a band pass filter to remove mains hum at 50 and 60 Hz. When the magnitude of this high frequency component crosses a certain threshold, the stimulator 100 assumes that EMG contamination is occurring and a red light is used to warn the user that he or she is to reduce muscle tension in the forehead or jaw. The red light is also displayed when the breathing rate acquired from the breathing sensor 1002 surpasses a certain threshold. When both conditions are false, the visual stimulus is a blue light which pulsates according to the user's heart rate, the heart rate being acquired from the pulse oximeter 106.

An ideal wake time to begin gradual light stimulation of a sleeping user can be determined by analyzing the EEG signal. REM sleep is seen as a shallow sleep phase, and when it occurs near a predetermined waking time it can be used as an indicator of the ideal wake time. REM sleep is easily detected from the sleep EEG spectrogram because it includes no sleep spindles, high beta activity, and no delta and alpha activity.

Other modifications and implementations will occur to those skilled in the art without departing from the spirit and the scope of the invention as claimed. Accordingly, the above description is not intended to limit the invention except as indicated in the following claims.

What is claimed is:

1. A forehead-wearable light stimulator comprising:
one or more physiological sensors capable of acquiring physiological parameters of a wearer of the light stimulator;
a central processing unit capable of receiving the physiological parameters from the one or more physiological sensors, and accordingly providing a light stimulation signal at times and intensities responsive to the physiological parameters;
at least one light source capable of receiving the light stimulation signal, and accordingly emitting light; and
one or more light pipes capable of conducting the light from the at least one light source to the eyes of the wearer of the light stimulation apparatus.

2. The apparatus of claim 1, wherein the one or more physiological sensors include at least one of:
an EEG sensor; a pulse oximetry sensor; a heart rate sensor; a breathing sensor; and a temperature sensor.

3. The apparatus of claim 1, wherein the light stimulation signal is obtained by transforming one or more physiological parameters.

4. The apparatus of claim 1, also comprising:
a dream onset detector, capable of analyzing the physiological parameters so as to provide a dream onset signal,
and wherein the central processing unit provides the light stimulation signal in response to the dream onset signal, so as to induce lucid dreaming.

5. The apparatus of claim 1, also comprising a waking time determiner, capable of determining an ideal waking time, and wherein the luminous stimulation signal is provided at the ideal waking time so as to awaken the wearer.

6. The apparatus of claim 1, wherein the at least one light source is a multi-color light source.

7. The apparatus of claim 1, wherein the at least one light source is substantially of a single color.

8. A forehead-wearable light stimulator comprising:
an EEG sensor capable of acquiring EEG parameters of a wearer of the light stimulator;
a central processing unit capable of receiving the EEG parameters from the EEG sensors, and accordingly providing a light stimulation signal at times and intensities responsive to the EEG parameters;
at least one light source capable of receiving the light stimulation signal, and accordingly emitting light; and
one or more light pipes capable of conducting the light from the at least one light source to the eyes of the wearer of the light stimulation apparatus.

9. The apparatus of claim 8, wherein the EEG sensor is cooperative with at least one of:
a pulse oximetry sensor; a heart rate sensor; a breathing sensor; and a temperature sensor.

10. The apparatus of claim 8, wherein the light stimulation signal is obtained by analyzing an EEG signal.

11. The apparatus of claim 8, also comprising:
a dream onset detector, capable of analyzing the EEG signal so as to provide a dream onset signal,
and wherein the central processing unit provides the light stimulation signal in response to the dream onset signal, so as to induce lucid dreaming.

12. The apparatus of claim 8, also comprising a waking time determiner, capable of determining an ideal waking time, and wherein the luminous stimulation signal is provided at the ideal waking time so as to awaken the wearer.

13. The apparatus of claim 8, wherein the at least one light source is a multi-color light source.

14. The apparatus of claim 1, wherein the at least one light source is substantially of a single color.
